(19) United States
(12) Patent Application Publication
(10) Pub. No.: US 2024/0161307 A1
(43) Pub. Date: May 16, 2024

(54) PREDICTING VELOCIMETRY USING MACHINE LEARNING MODELS

(71) Applicants: NANYANG TECHNOLOGICAL UNIVERSITY, Singapore (SG); MASSACHUSETTS INSTITUTE OF TECHNOLOGY, cAMBRIDGE, MA (US); BROWN UNIVERSITY, Providence, RI (US)

(72) Inventors: Ming Dao, Cambridge, MA (US); Subra Suresh, Singapore (SG); George Karniadakis, Providence,, RI (US); Shengze Cai, Providence, RI (US); He Li, Providence, RI (US)

(21) Appl. No.: 18/282,679

(22) PCT Filed: Mar. 17, 2022
(86) PCT No.: PCT/US2022/020743

§ 371 Date: Sep. 18, 2023

Related U.S. Application Data
(60) Provisional application No. 63/162,780, filed on Mar. 18, 2021.

(51) Int. Cl. G06T 7/20; G06T 7/00

(52) U.S. Cl. CPC G06T 7/20; G06T 7/0012; G06T 2207/10016; G06T 2207/20081; G06T 2207/20084; G06T 2207/30104

(57) ABSTRACT

Methods and systems for estimating fluid flow characteristics are provided. In one embodiment, a method is provided that includes receiving a plurality of images of fluid flow at a plurality of times. The images may be analyzed with a machine learning model to predict one or more physical characteristics of the fluid flow, such as a velocity field, a pressure field, and/or a stress field. A loss measure may be calculated for the physical characteristics based on physical fluid flow constraints, boundary condition constraints, and/or data mismatch constraints. The machine learning model may be updated based on the loss value.

### CROSS-REFERENCE TO RELATED APPLICATIONS

The present application claims priority to U.S. Provisional Patent Application No. 63/162,780, filed on Mar. 18, 2021, the disclosure of which is incorporated herein by reference for all purposes.

### GOVERNMENT RIGHTS

This invention was made with government support under grant number R01 HL154150 awarded by the National Institutes of Health and grant number DE-500019453 awarded by the U.S Department of Energy. The government has certain rights in the invention.

### BACKGROUND

Human blood, primarily comprising plasma, red blood cells (RBCs), white blood cells and platelets, is a non-Newtonian fluid exhibiting shear-thinning behavior. The effect of this non-Newtonian behavior becomes more pronounced in microcirculation. Understanding and quantifying the biorheology of blood is essential for gaining insights into the mechanisms that influence microcirculation in physiology and disease. The characteristics of hemodynamics also determine the vascular integrity and blood cell transport in physiology, e.g., the margination of platelets. Platelet margination refers to the phenomenon of formation of a cell-free layer near the vessel wall in blood flow, as RBCs accumulate in the center of the vessel. Compromised hemodynamics can result in pathologies such as endothelial cell inflammation and dysfunction, undesired platelet activation and the formation of clots within a blood vessel. Fluid flow may also impact other applications (e.g., drag coefficients for vehicles, surface flow dynamics for bodies of water, and cooling of liquids).

### SUMMARY

The present disclosure presents new and innovative systems and methods for estimating fluid flow characteristics. In one aspect, a method is provided that includes receiving a plurality of microfluidic images of blood flow within a blood vessel at a plurality of times and analyzing, with a machine learning model, the plurality of microfluidic images to predict at least two fields for predicted blood flow within the blood vessel. The at least two fields may be selected from the group consisting of a velocity field, a pressure field, and/or a stress field. The method may also include calculating a loss measure for the at least two fields based on at least two of physical fluid flow constraints, boundary condition constraints for blood flow within the blood vessel, and data mismatch constraints between the predicted blood flow and the plurality of microfluidic images. The method may further include updating the machine learning model based on the loss value.

In a second aspect according to the first aspect, the at least two fields are two-dimensional fields for the predicted blood flow.

In a third aspect according to any of the first and second aspects, the at least two fields are three-dimensional fields for the predicted blood flow.

In a fourth aspect according to any of the first through third aspects, the boundary condition constraints include a boundary condition measure computed to measure compliance of the predicted blood flow with a predetermined boundary condition.

In a fifth aspect according to the fourth aspect, the predetermined boundary condition is selected from the group consisting of a slip boundary condition and a non-slip boundary condition.

In a sixth aspect according to any of the first through fifth aspects, the physical fluid flow constraints include a physical conservation measure computed to measure compliance of the predicted blood flow with fluid dynamic flow constraints.

In a seventh aspect according to the sixth aspect, the fluid dynamic flow constraints include an optical flow constraint.

In an eighth aspect according to any of the sixth and seventh aspects, the physical conservation measure is computed at a predetermined set of coordinates within the predicted blood flow.

In a ninth aspect according to any of the first u h ninth aspects, the machine learning model is a fully-connected neural network.

In a tenth aspect according to any of the first through ninth aspects, the microfluidic images are two-dimensional images of the blood vessel.

In an eleventh aspect according to any of the first through tenth aspects, the microfluidic images are three-dimensional images of the blood vessel.

In a twelfth aspect according to any of the first through eleventh aspects, the microfluidic images are successive images captured by a video camera.

In a thirteenth aspect according to any of the first through the microfluidic images depict at least one of individual blood vessels and/or individual platelets within the blood vessel.

In a fourteenth aspect a system is provided that includes a processor and a memory storing instructions. When executed by the processor, the instructions may cause the processor to receive a plurality of microfluidic images of blood flow within a blood vessel at a plurality of times and analyze, with a machine learning model, the plurality of microfluidic images to predict at least two fields for predicted blood flow within the blood vessel, the at least two fields selected from the group consisting of a velocity field, a pressure field, and/or a stress field. The instructions may further cause the calculate a loss measure for the at least two fields based on at least two of physical fluid flow constraints, boundary condition constraints for blood flow within the blood vessel, and data mismatch constraints between the predicted blood flow and the plurality of microfluidic images and update the machine learning model based on the loss value.

In a fifteenth aspect according to the fourteenth aspect, the at least two fields are two-dimensional fields for the predicted blood flow.

In a sixteenth aspect according to any of the fourteenth and fifteenth aspects, the at least two fields are three-dimensional fields for the predicted blood flow.

In a seventeenth aspect according to any of the fourteenth through sixteenth aspects, the boundary condition constraints include a boundary condition measure computed based on compliance of the predicted blood flow with a predetermined boundary condition.

In an eighteenth aspect according to the seventeenth aspect, the predetermined boundary condition is selected from the group consisting of a slip boundary condition and a non-slip boundary condition.

In a nineteenth aspect according to any of the fourteenth through eighteenth aspects, the physical fluid flow constraints include a physical conservation measure computed based on compliance of the predicted blood flow with fluid dynamic flow constraints.

In a twentieth aspect according to the nineteenth aspect, the fluid dynamic flow constraints include an optical flow constraint.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIG. 1 illustrates a system according to an exemplary embodiment of the present disclosure.

FIG. 2 illustrates a training procedure according to an exemplary embodiment of the present disclosure.

FIG. 3 illustrates a method according to an exemplary embodiment of the present disclosure.

FIGS. 4A-4J illustrate experimental results for predicted fluid flow fields according to exemplary embodiments of the present disclosure.

FIG. 5 illustrates a computer system according to an exemplary embodiment of the present disclosure.

### DETAILED DESCRIPTION OF EXAMPLE EMBODIMENTS

Scientific research over the past several decades has led to rapid advances in in vivo imaging techniques. Despite this progress, it is currently not feasible to observe in real time many in vivo biological processes in microcirculation, such as the rupture of a microaneurysm (MA) in the retinal microvasculature and the initiation and development of blood clots. To compensate for this void in the ability to track the origins and progression of disease states, in vitro experiments of blood flow within microfluidic channels have been developed to mimic in vivo circulation under both physiologically and pathologically relevant conditions. Microfluidic devices and lab-on-a-chip platforms offer advantages in exploring the biophysical and biochemical characteristics of blood flow in microvessels. Benefits of these devices include the need for only small volumes of blood for analysis and precise control over temperature, concentrations of gas and chemicals in the blood. Another distinct advantage of such microfluidic platforms is that they enable quantitative determination of various key parameters associated with hemodynamics, such as spatial distributions of velocity and stress fields, under well-controlled experimental conditions so that mechanistic insights could be extracted for transitions from healthy to pathological states.

A wide variety of experimental techniques is currently available to assess the hemodynamics of in vitro blood flow in microcirculation. The state of the art optical whole-field velocity measurement technique is micro-particle image velocimetry (μPIV), a non-intrusive method used to estimate flow fields in microchannels. Various algorithms employing μPIV have been well developed in recent years and this technology has been successfully applied to a broad range of biological problems, μPIV can provide measurements of blood velocity along channels in microcirculation, with high spatial and temporal resolution, by analyzing the motion of laser-induced fluorescence tracers seeded into blood. However, the experimental apparatus requires elaborate calibration and may not be amenable for wide or easy deployment. Other approaches to monitor flow motion, such as advanced PIV methods or optical flow monitoring techniques are able to quantify hemodynamics from images of blood flow in the microchannels using RBCs and platelets as tracers, thereby requiring less hardware. However, their accuracy in providing near-wall flow measurements, which is critical for inferring the pathogenic basis of blood rheology, and the estimation of wall shear stress, could be compromised owing to the formation of cell-free layers in the vicinity of blood vessel walls.

Computational fluid dynamics (CFD) models have also been employed to simulate blood flow in micro-vessels or channels to investigate the pathophysiology of circulatory diseases. By invoking laws of physics (e.g., Navier-Stokes equations) and specific boundary conditions (such as no-slip conditions at the blood vessel wall), CFD models can simulate the flow field and extract key hemodynamic indicators. Several studies have employed CFD models to compute flow and stress fields in normal microvessels as well as channels with various shapes, such as stenotic channels (in which constricted flow from plaques markedly alter flow characteristics), aneurysmal vessels containing a bulge in the vessel as a result of a weakened vessel wall and other vasculatures with complex geometries. However, results extracted from CFD models are very sensitive to the flow boundary conditions assumed at the inlets and outlets, which can be patient-specific. Even moderate errors in flow boundary conditions could lead to large uncertainty in the estimation of the flow fields. In addition, CFD simulations could be computationally cumbersome for modeling flow fields with moving boundaries or geometric variation such as the hemodynamics changes due to accumulation of blood cells.

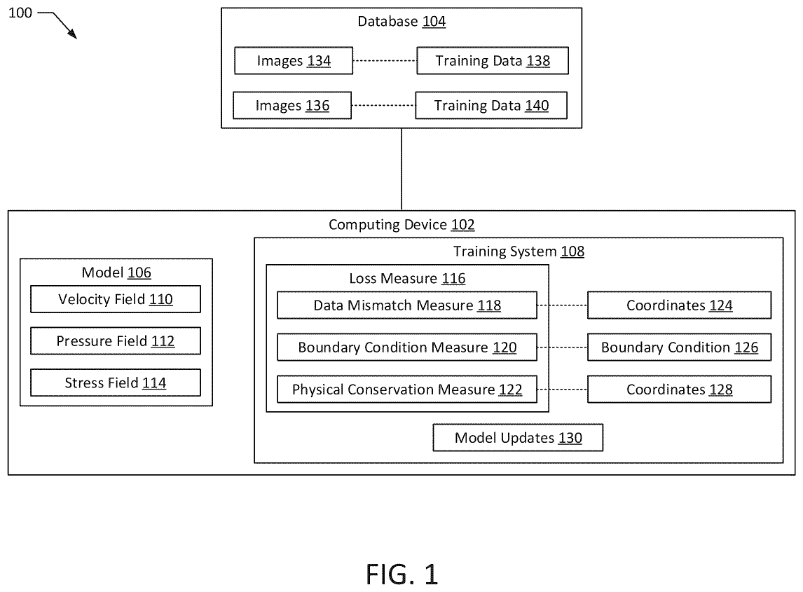

FIG. 1 illustrates a system 100 according to an exemplary embodiment of the present disclosure. The system 100 may be configured to train and update machine learning models to predict fluid velocimetry to address the above-discussed issues with existing techniques for fluid velocimetry measurement. For example, the system 100 may be configured to train a model 106 that can be used to predict velocity, pressure, and/or stress fields for fluid flow within a channel (e.g., a blood vessel). The system 100 includes a computing device 102 and a database 104. The computing device 102 may be configured to train or update the model 106 using a training system 108. In particular, the computing device 102 may receive images 134, 136 and training data 138, 140 from the database 104 for use in training the model 106.

In particular, the database 104 may store images 134, 136 associated with training data 138, 140. The images 134, 136 may depict fluid flow, such as blood flow, through a fluid channel, such as a blood vessel. In particular, the images 134, 136 may have a high enough resolution to depict individual particles (e.g., blood cells) within a depicted fluid flow. Each of the images 134, 136 may contain multiple images of the same area over time, showing a change in flowing fluid over time. For example, each of the images 134, 136 may contain multiple images of the same blood vessel (e.g., 10 images, 50 images, 100 images, or more) at different points in time. In certain implementations, the images 134, 136 may be microfluidic images captured by a video camera and may depict individually discernible particles (e.g., blood cells and/or platelets). In particular, the images 134, 136 may include still images of fluid flow over time (e.g., two-dimensional images, three-dimensional images) and/or video of fluid flow over time (e.g., two-dimensional video, three-dimensional video). The training data 138, 140 may include experimentally verified flow information for the fluid flow depicted within the images 134, 136. For example, the training data 138, 140 may contain an experimentally measured velocity field, pressure field, and/or stress field for the flow depicted within the images 134, 136. In certain implementations, the training data 138, 140 may be experimentally measured using techniques such as optical flow techniques, fluid flow simulations, particle image velocimetry, and direct measurement (e.g., using electric, magnetic, and/or acoustic sensors). Additionally or alternatively, the training data 138, 140 may include one or more of a computational domain (e.g., spatial coordinates within the images 134, 136 in which fluid flow is calculated) and/or location points for loss measure calculations (e.g., the coordinates 124, 128), as discussed further below.

The computing device 102 may receive images 134, 136 and training data 138, 140 from the database 104 for use in training the model 106. The model 106 may be configured to generate one or more fields representative of a predicted flow within the images 134, 136. As a specific example, the model 106 may receive images 134 depicting blood flow within a blood vessel. The model 106 may be configured to predict one or more physical parameters of the fluid flow within the fluid channel. For example, the model 106 may be configured to predict one or more of a velocity of the blood flow at one or more locations within the fluid channel, pressure at one or more locations within the fluid channel, and stress (e.g., shear stress) at one or more locations within the fluid channel. In particular, the model 106 may generate one or more of a velocity field 110 representative of the velocity of a predicted fluid flow at one or more locations within the fluid channel, a pressure field 112 representative pressure within a predicted fluid flow, and/or a stress field representative of shear stress caused by a predicted fluid flow.

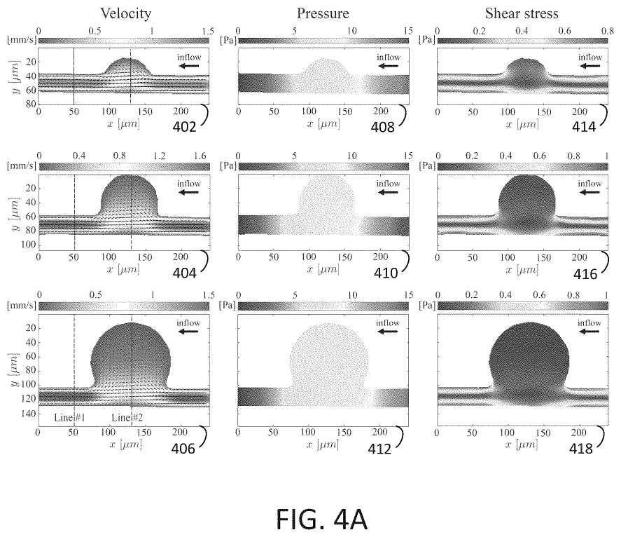

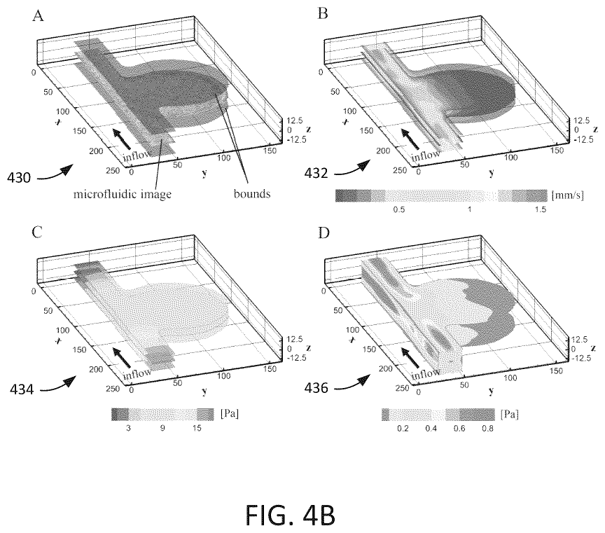

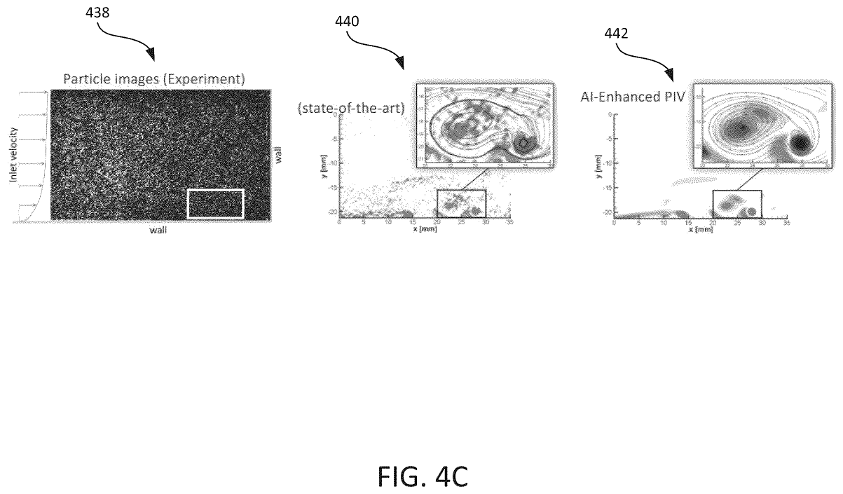

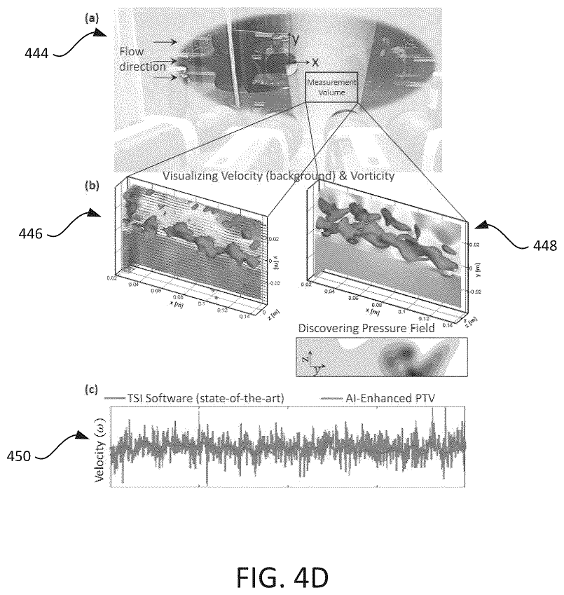

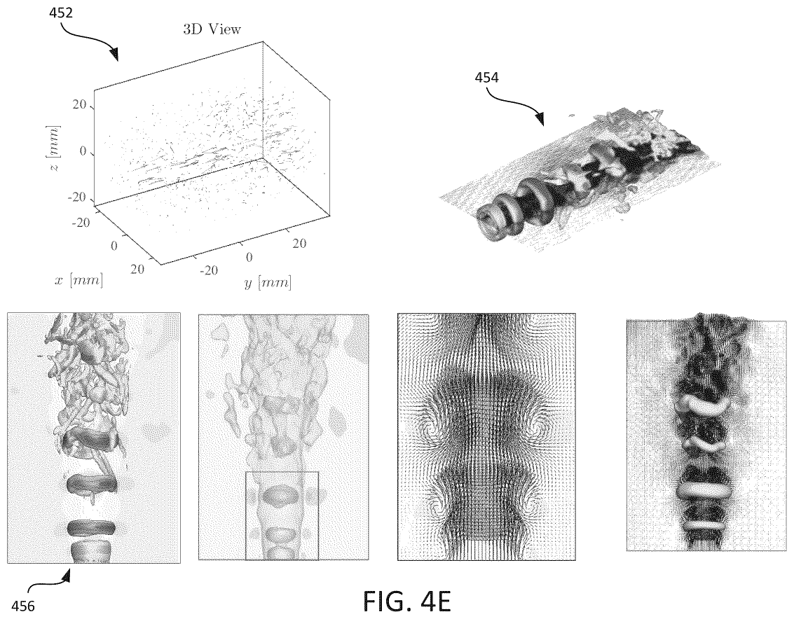

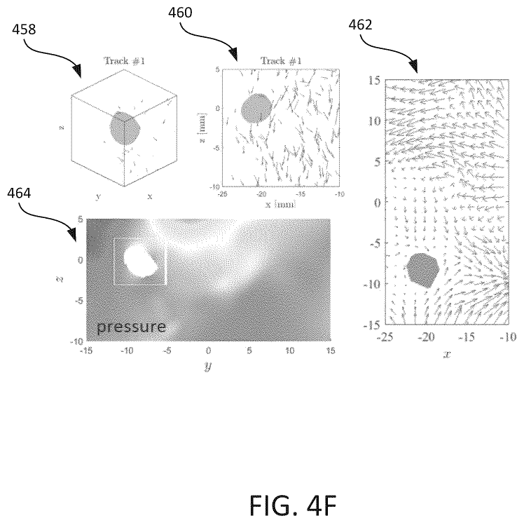

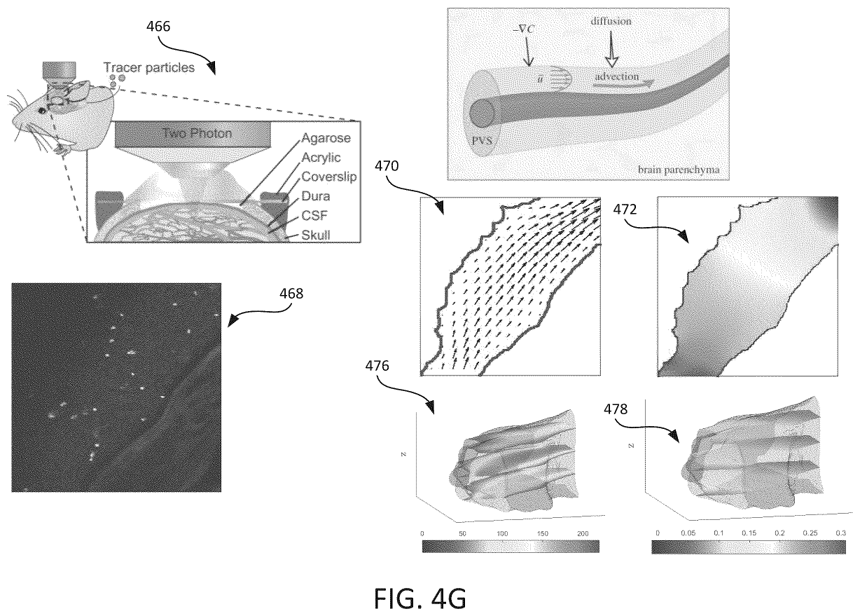

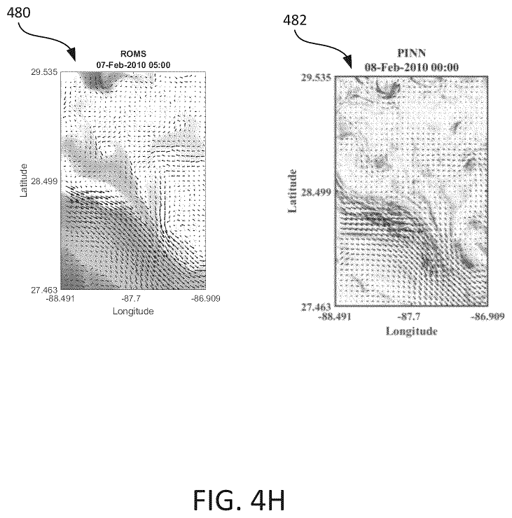

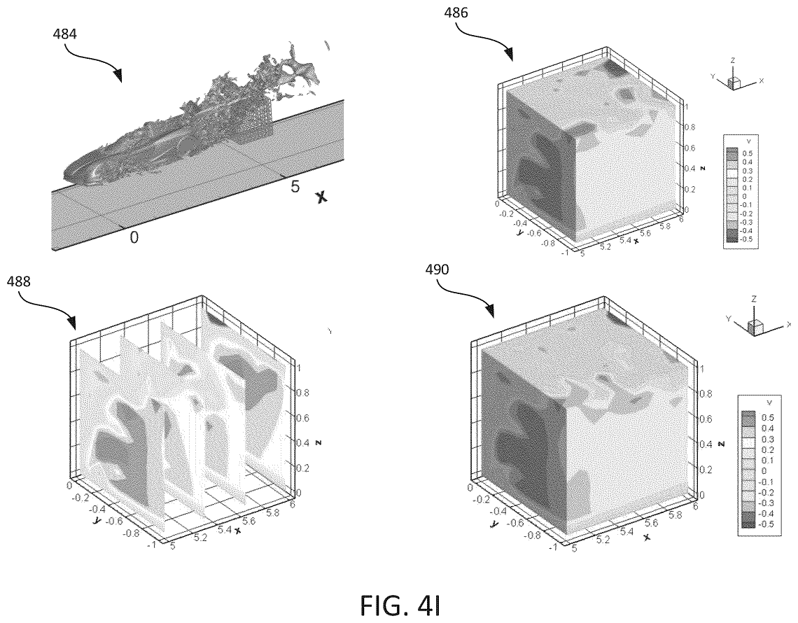

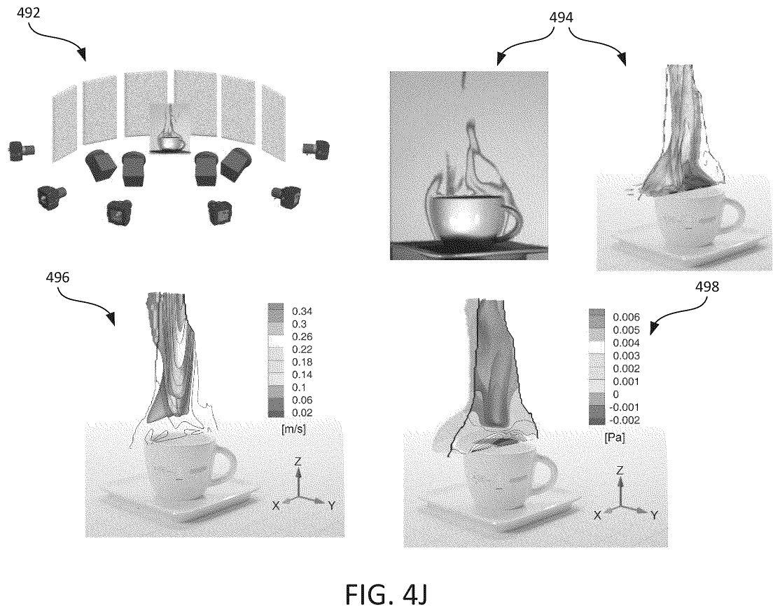

The fields 110, 112, 114 may be either two-dimensional or three-dimensional. For example, the images 134, 136 may depict a two-dimensional view of fluid flow within a fluid channel. The fields 110, 112, 114 may be generated to include predicted flow velocity, flow pressure, and/or shear stress at the locations within the fluid channel depicted within the images 134, 136. Because the images 134, 136 depict a two-dimensional view, the resulting fields 110, 112, 114 in such an implementation may correspondingly depict a two-dimensional view of the predicted velocities, pressures, and/or stresses. In additional or alternative implementations, the model 106 may be configured to further extend a predicted two-dimensional field based on predicted depth information for a depicted fluid flow (e.g., within a depicted fluid channel). Exemplary two-dimensional and three-dimensional fields are depicted in FIGS. 4A and 4B, discussed below.

In order to accurately predict the velocity, pressure, and/or stress fields 110, 112, 114, the model 106 may be trained to incorporate and comply with underlying laws of physics. For example, the model 106 may be trained to comply with typical fluid flow constraints, such as the optical flow constraint and boundary conditions where flow within a channel (e.g., a blood vessel) interacts with the boundaries of the channel. Depending on the type of fluid flow depicted within the images 134, 136, the boundary conditions selected for the model 106 may be selected between slip conditions and non-slip conditions. For example, due to the non-Newtonian characteristics of blood flowing through a blood vessel, the model 106 may be trained to incorporate non-slip boundary conditions. As another example, for other types of fluid flows (e.g., for Newtonian fluids), the model 106 may be trained to incorporate slip boundary conditions. As will be appreciated by one skilled in the art, there are multiple constraints and mechanisms used to model fluid flow within channels. Depending on the implementation (e.g., different sizes of channels, different fluid viscosities, other fluid characteristics), various other fluid flow constraints may be used to train the model 106. All such changes to the discussed implementations are considered within the scope of the present disclosure.

To ensure that the model 106 accurately incorporates these underlying laws of physics, the training system 108 may be configured to determine when a predicted fluid flow from the model 106 deviates from physically possible flows and to disincentivize such predictions. In particular, the training system 108 may be configured to calculate a loss measure 116 based at least in part on deviations from physically possible fluid flows. For example, the loss measure 116 may be calculated based on a data mismatch measure 118, a boundary condition measure 120, and a physical conservation measure 122. In particular, the loss measure 116 may be calculated as a weighted combination of the measures 118, 120, 122. The boundary condition measure 120 and the physical conservation measure 122 may be calculated based on deviations from expected fluid flows that comply with the underlying laws of physics. In particular, the boundary condition measure 120 may be configured to measure deviations of the fields 110, 112, 114 from a predetermined boundary condition 126. Depending on the type of fluid depicted in the images 134, 136 and/or the size of the fluid channel (or other fluid flow conditions), the boundary condition 126 may be selected from among a slip boundary condition and a non-slip boundary condition. Furthermore, the physical conservation measure 122 may be calculated to measure deviations of the fields 110, 112, 114 from fluid flow constraints representative of physically possible fluid flow conditions, such as the optical flow constraint. The physical conservation measure 122 may be calculated at multiple coordinates 128 within the velocity field 110, pressure field 112, and/or stress field 114 output by the model 106. For example, a random set of spatial coordinates 128 within the images 134, 136 analyzed by the model 106 may be selected prior to generating the fields 110, 112, 114. Once generated, corresponding coordinates 128 within the fields 110, 112, 114 may be analyzed for deviation from the fluid flow constraints in order to generate the physical conservation measure 122.

The loss measure 116 may further be calculated to incorporate a data mismatch measure 118. The data mismatch measure 118 may be computed to measure deviations of spatial information within the fields 110, 112, 114 produced by the model 106 from spatial information within the images 134, 136. Similar to the physical conservation measure 122, the data mismatch measure 118 may be calculated at a plurality of coordinates 124 within the fields 110, 112, 114. In particular, the data mismatch measure 118 may be calculated based on a random set of coordinates 124 within the field 110, 112, 114 and corresponding coordinates within the images 134, 136. In certain implementations, the coordinates 124 may be similar or identical to the coordinates 128. Additionally or alternatively, different coordinates 124 may be used for the data mismatch measure 118 from the coordinates 128 used for the physical conservation measure 122.

Once calculated, the data mismatch measure 118, boundary condition measure 120, and physical conservation measure 122 may be combined to form the loss measure 116 for the fields 110, 112, 114 generated by the model 106. In certain implementations, the loss measure 116 may be generated as a weighted combination of the data mismatch measure 118, the boundary condition measure 120, and the physical conservation measure 122. In certain implementations, one or more of the data mismatch measure 118, the boundary condition measure 120, and the physical conservation measure 122 may have a proportionally larger effect on the loss measure 116 (e.g., may have a larger weight). For example, in certain implementations, the data mismatch measure 118 and/or the physical conservation measure 122 may have a larger weight (and therefore larger effect on the loss measure 116) than the boundary condition measure 120. It should be understood that different implementations of the training system 108, the model 106, and/or the loss measure 116 may result in different weights being selected for the data mismatch measure 118, the boundary condition measure 120, and the physical conservation measure 122. Additionally or alternatively, certain implementations of the loss measure 116 may omit one or more of the data mismatch measure 118, the boundary condition measure 120, and the physical conservation measure 122. All such implementations are hereby considered within the scope of the present disclosure.

Based on the loss measure 116, the model 106 may be updated. For example, the training system 108 may generate model updates 130 for the model 106. In certain implementations, the model updates 130 may include changing the weights of one or more nodes within the model 106. Additionally or alternatively, model updates 130 may include adjusting the features analyzed by the model 106 (e.g., changing corresponding features for one or more nodes within the model 106).

One or both of the computing device 102 and the database 104 may be implemented by a computing system. For example, although not depicted, one or both of the computing device 102 and the database 104 may include a processor and a memory that implement at least one operational feature. For example, the memory may contain instructions which, when executed by the processor, cause the processor to perform one or more operational features of the computing device 102 and/or the database 104. Additionally, the computing device 102 and the database 104 may communicate using a network. For example, the computing device 102 and the database 104 may communicate with the network using one or more wired network interfaces (e.g., Ethernet interfaces) and/or wireless network interfaces (e.g., Wi-Fi®, Bluetooth®, and/or cellular data interfaces). In certain instances, the network may be implemented as a local network (e.g., a local area network), a virtual private network, L1, and/or a global network (e.g., the Internet). In additional or alternative implementations, the database 104 may be implemented at least in part by the computing device 102.

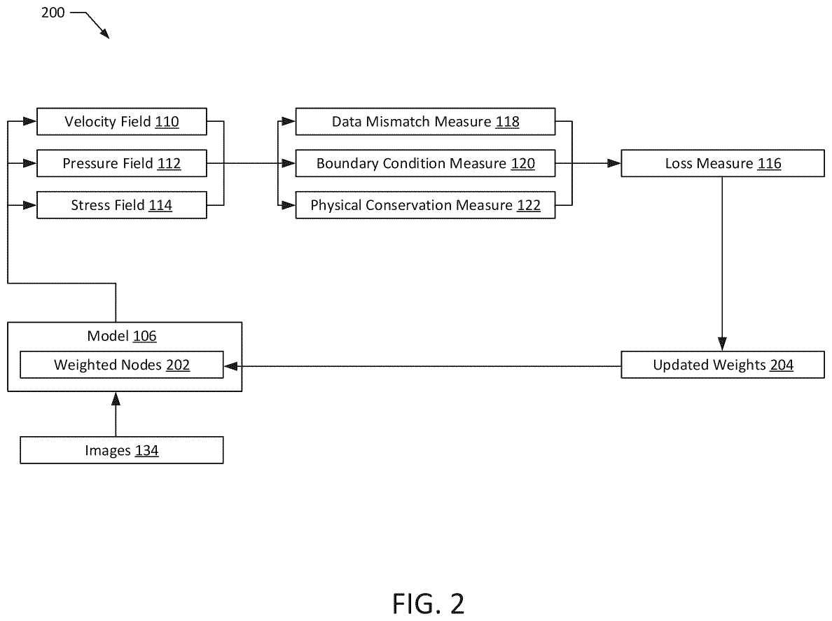

FIG. 2 illustrates a training procedure 200 according to an exemplary embodiment of the present disclosure. The training procedure 200 may represent an exemplary training round for the model 106 performed by the computing device 102 and the training system 108. In particular, the training procedure 200 may be performed to update the model based on images 134 of fluid flowing through a fluid channel that are stored within the database 104. The images 134 may be received from the database 104 along with corresponding training data 138. For example, the images 134 may include 100 sequential images of fluid flow through a segment of fluid channel.

The images 134 may then be provided to the model 106, which may analyze the images 134 to generate a velocity field 110, a pressure field 112, and a stress field 114. The velocity field 110 may include velocity estimates for the fluid flowing through the fluid channel at multiple locations within the segment of the fluid channel. The pressure field 112 may similarly include pressure estimates for the fluid flowing through the fluid channel in multiple locations. The stress field 114 may include shear stress estimates for the fluid flowing near (e.g., within a predetermined distance of) edges of the fluid channel.

The model 106 may be implemented as a machine learning model configured to analyze multiple sequential images of fluid flow to generate the velocity, pressure, and stress fields 110, 112, 114. For example, the model 106 may be implemented as a neural network (e.g., a fully-connected neural network) formed from a plurality of interconnected weighted nodes 202. In one implementation, the model 106 may be formed from a 10-layer neural network with 80 neurons per layer. Such an implementation may be suited to generating two-dimensional fields 110, 112, 114. In additional or alternative implementations, the model 106 may be formed from a 10-layer neural network with 100 neurons per layer, which may be suited to generating three-dimensional fields 110, 112, 114. Each of the nodes 202 may incorporate or correspond to different aspects or features of the plurality of images 134 in order to form the fields 110, 112, 114.

The training system 108 may then receive the velocity, pressure, and stress fields 110, 112, 114 generated by the model 106 and may use these fields 110, 112, 114 to generate a loss measure 116. As explained above, the data mismatch measure 118 may be calculated to measure deviations of data values at the coordinates 124 within the fields 110, 112, 114 produced by the model 106 and data values at the coordinates 128 within the images 134, 136. For example, the data mismatch measure 118 may be calculated as:

$${ℒ}_{data} = \frac{1}{{N}_{d}} {\sum }_{i = 1}^{{N}_{d}} ‖ {I}_{data} ( {x}_{i} , {t}_{i} ) - {I}_{NN} ( {x}_{i} , {t}_{i} ) ‖$$

where:
[symbol] _{data} is the data mismatch measure,
I_{data} is the intensity (e.g., magnitude) of the data value from the analyzed images (e.g., the images 134) at coordinate xᵢ and time tᵢ,
I_{NN} is the intensity (e.g., magnitude) of the data value from the output the model 106 (e.g., at least one of the fields 110, 112, 114) at coordinate xᵢ and time tᵢ,
N_{d} is the number of pixels analyzed in the image and predicted fields (e.g., the number of coordinates 124),
xᵢ is the coordinate location of the i-th pixel, and
tᵢ is the time of the i-th pixel (e.g., a time stamp for the image containing the i-th pixel, a frame number for the image containing the i-th pixel).

The boundary condition measure 120 may be calculated differently based on the selected boundary condition 126. For example, the boundary condition measure 120 may be calculated for a slip boundary condition and/or for a no-slip boundary condition. In the case of images 134 of blood flow within a blood vessel, a no-slip boundary condition may be selected due to the fluid characteristics of blood flow, as explained above. In such instances, the boundary condition measure 120 may be calculated as:

$${ℒ}_{bcs} = \frac{1}{{N}_{b}} {\sum }_{i = 1}^{{N}_{b}} ‖ u ( \partial Ω ) ‖$$

where:
[symbol] _{bcs} is the boundary condition measure 120,
N_{b} is the number of pixels analyzed along the border in the predicted fields (e.g., the number of border coordinates), and
∂Ω is the boundary condition at the analyzed pixel location.

The physical conservation measure 122 may be calculated to ensure that, at various coordinates 128 within the fields 110, 112, 114, the predicted velocity, pressure, and/or stress values comply with physical constraints on fluid flow. For example, the physical conservation measure 122 may be calculated as:

$${ℒ}_{res} = \frac{1}{{N}_{e}} {\sum }_{i = 1}^{{N}_{e}} {\sum }_{j = 1}^{S} ‖ {e}_{j} ( {x}_{i} , {t}_{i} ) ‖$$

where:
[symbol] ᵣₑₛ is the boundary condition measure,
Nₑ is the number of pixels analyzed in the predicted fields (e.g., the number of coordinates 128)
eⱼ is the boundary condition residuals for the no-slip boundary conditions, namely:

e₁=Iₜ+uIₓ+vI_{y}

e₂,e₃,e₄=uₜ(u·∇)u+∇p−∇·(μ(∇u+(∇u)^{T}))

e₅=uₓ+v_{y}+w_{z},
xᵢ is the coordinate location of the i-th pixel, and
tᵢ is the time of the i-th pixel (e.g., a time stamp for the image containing the i-th pixel, a frame number for the image containing the i-th pixel).

Once calculated, the data mismatch measure 118, boundary condition measure 120, and physical conservation measure 122 may be combined to form the loss measure 116. In particular, as explained above, the loss measure 116 may be a weighted combination of the measures 118, 120, 122, such as:

[symbol]

=λ_{d} [symbol] _{data}+λ_{b} [symbol] _{bcs}+ [symbol] ᵣₑₛ

where:
[symbol] is the loss measure 116,
λ_{d} is the weight for the data mismatch measure 118, and
λ_{b} is the weight for the boundary condition measure 120.
   Large values of λ_{d} and λ_{b} may accelerate training of the model 106, but may also result in overfitting of the training data ; reducing overall accuracy.

The model 106 may then be updated based on the loss measure 116. For example, one or more updated weights 204 may be determined based on the loss measure 116 (e.g., may be randomly altered, may be selected as a weighted combination of previous values). The updated weights 204 may then be added to the model 106 for future use. Procedures similar to the procedure 200 may then be repeated in order to train the model 106 to accurately predict velocity, pressure, and/or stress fields for a depicted fluid flow.

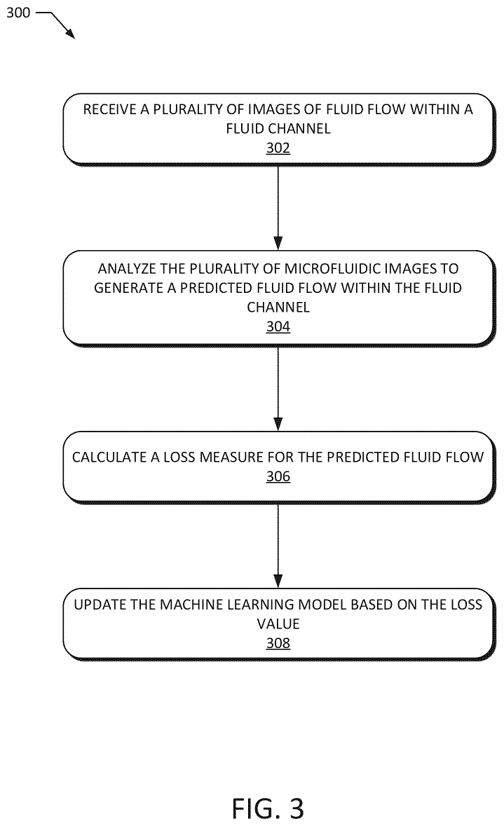

FIG. 3 illustrates a method 300 according to an exemplary embodiment of the present disclosure. The method 300 may be performed to train a model to predict fluid flow conditions within a fluid channel based on a series of images depicting the fluid flow. The method 300 may be implemented on a computer system ; such as the system 100. For example, the method 300 may be implemented by the computing device 102. The method 300 may also be implemented by a set of instructions stored on a computer readable medium that, when executed by a processor, cause the computer system to perform the method 300. For example, all or part of the method 300 may be implemented by a processor and/or memory of the computing device 102. Although the examples below are described with reference to the flowchart illustrated in FIG. 3, many other methods of performing the acts associated with FIG. 3 may be used. For example, the order of some of the blocks may be changed, certain blocks may be combined with other blocks, one or more of the blocks may be repeated, and some of the blocks described may be optional.

The method 300 may begin with receiving a plurality of images of fluid flow within a fluid channel (block 302). For example, the computing device 102 may receive a plurality of images 134 that depict fluid flow within the fluid channel (e.g., blood flow within a blood vessel). The images 134 may be received as sequential images (e.g., a video file) depicting fluid flow through a segment of a fluid channel. In certain implementations, the images 134 may be microfluidic images of the fluid flow.

The plurality of images may be analyzed to generate a predicted fluid flow within the fluid channel (block 304). The images 134 may be analyzed using a machine learning model 106 to predict one or more physical characteristics of fluid flow within the fluid channel depicted in the images 134. For example, the model 106 may generate one or more of a velocity field 110, a pressure field 112, and/or a stress field 114 for shear stresses for the fluid flow within the depicted fluid channel.

A loss measure may be calculated for the predicted fluid flow (block 306). For example, a loss measure 116 may be calculated based on one or more of a data mismatch measure 118, a boundary condition measure 120, and/or a physical conservation measure 122, as discussed above. One or more of the measures 118, 120, 122 may be calculated for various coordinates 124, 128 within the depicted fluid channel and/or the images 134.

The machine learning model may be updated based on the loss value (block 308). For example, model updates 130 (e.g., updated weights 204) may be generated based on the loss measure 116 for the model 106. Model updates 130 may then be applied to update one or more nodes of the model 106 and may be used in future analyses performed by the model 106.

Although discussed in the singular, in certain implementations, the method 300 may be performed to analyze more than one set of images depicting more than one fluid channel. For example, at block 302, multiple sets of images of multiple fluid channels may be received. In such instances, blocks 304, 306 may be repeated for each of the received sets of images. Furthermore, the model updates 130 may not be generated for each individual set of images analyzed by the model 106. Instead, multiple image sets may be analyzed before the model updates 130 are generated. The method 300 may also be repeated multiple times to train a model 106. For example, the method 300 may be repeated multiple times for multiple sets of images from the database 104.

In this way, the method 300 enables the training of a model 106 that can accurately predict the fluid flow characteristics of fluid flowing within a fluid channel based on images of the fluid flowing through the channel. Such techniques may enable improved velocimetry and can seamlessly integrate with in vivo and in vitro data to accurately measure blood flow within a patient with greater accuracy and with reduced measurement time, as specialized measurement techniques like particle image velocimetry are not required.

Furthermore, many of the examples discussed above concern images depicting the flow of blood cells and other particles through a blood vessel. In practice, however, similar techniques may be used with images of fluid flow through other channels (e.g., other tubes or pipes) so long as passive scalars (e.g., particles, objects, temperature, cells) are discernible within the images the images are taken with a high enough frequency. For example, these techniques may be used to predict fluid flow for Newtonian and non-Newtonian fluids. As another example, these techniques may be used to predict fluid flow for liquids and/or other types of fluids (e.g., gases). In still further instances, these techniques may be used to predict two-dimensional and/or three-dimensional fluid flows. As some specific examples, fluid flows may be predicted for one or more of blood flow (e.g., within a body's circulatory system), water flow (e.g., horseshoe vortexes, water jets, movement of bubbles within water, sea surface currents), cerebrospinal fluid (CSF), and air flow (e.g., behind an aircraft, behind a vehicle, behind an animal). Several of these examples are discussed in greater detail below.

Regarding blood flow, FIGS. 4A-4B illustrate predicted blood flow fields according to an exemplary embodiment of the present disclosure. FIG. 4A depicts two-dimensional velocity fields 402, 404, 406, pressure fields 408, 410, 412, and stress fields 414, 416, 418. The fields were generated using a model trained using the above-discussed techniques based on blood flow through channels with different sized protrusions representative of microaneurysms of varying sizes within a blood vessel.

As explained above, models may also be trained to infer three-dimensional information from two-dimensional images. In particular, the flow represented in the two-dimensional images may be extended according to the same fluid flow and boundary condition constraints (e.g., based on an estimated size of the blood vessel) to include changes in velocity, pressure, and/or shear stress at different depths within the blood vessel. For example, given a known depth for a particular channel of fluid flow (e.g., a blood vessel), the model 106 may be trained to generate three-dimensional fields 110, 112, 114 within the known depth. In certain implementations, the model 106 may be trained to generate three-dimensional fields instead of two-dimensional fields by adding additional nodes to each layer of the neural network. Additionally or alternatively, the loss measure may need to be updated (e.g., to include additional coordinates 124 for the data mismatch measure 118). FIG. 4B depicts an exemplary three-dimensional field generation process. In plot 430, a depth of the channel is estimated to establish the three-dimensional domain for subsequent calculations. Within this domain, the model then generates a velocity field 432, a pressure field 434, and a shear stress field 434.

In experimental testing using a modeled microaneurysm on a chip and in electroosmotic flow, the above-described techniques outperformed previous state of the art techniques (e.g., PIV and micro-PIV). In particular, the above-described techniques produce similar results to PIV and micro-PIV, but without requiring the cumbersome and invasive steps those processes require. The predicted velocity field also track with experimental results verified using platelet tracking, demonstrating that these techniques are capable of accurately predicting flow within blood vessels and other similar channels. Further details regarding these tests are presented in Artificial intelligence velocimetry for biomedical and engineering applications, Shengze Cai, He Li, Ming Dao, George Em Karniadakis, and Subra Suresh. This paper was attached as an Appendix to U.S. Provisional Patent Application No. 63/162,780 and is hereby incorporated by reference for all purposes.

Regarding horseshoe vortex flow, FIG. 4C depicts experimental results in which particle images 438 (e.g., images with a resolution of 1200×800) were captured for an experimentally-produced horseshoes flow within water. State-of-the-art PIV results 440 (e.g., with a resolution of 155×94) do not comply with the laws of physics, as flows are choppy and uneven. By contrast, the present techniques produced the results 442, which do.

Regarding 3D air wakes, FIG. 4D depicts experimental results to determine a 3D wake behind a falcon, shown in image 444. The state of the art results 446 are noisy and inaccurate, while the results 448 obtained by the present techniques remove the noise and improve the quality of the predicted flow. Comparison 450 further depicts the difference in noise between the results 446, 448.

Regarding 3D jet flow. FIG. 4E depicts experimental results to determine the flow within a 3D jet in water. Using a tomographic PTV setup with a fixed number (e.g., 3) cameras, the experimental results 454, 456 are able to improve on existing state of the art results 452.

Regarding movement of bubbles within a fluid, FIG. 4F depicts experimental results to determine velocity and pressure for a fluid flow as a bubble moves through water. The setup used 6 cameras to capture a bubble moving and deforming within turbulence. The state of the art results 458, 460 are inaccurate and only include velocity data. The present techniques are able to produce an improved velocity result 462 and are able to predict a pressure result 464 for the fluid flow.

Regarding CSF, FIG. 4G depicts experimental results in predicting the flow of CSF within the perivascular space of a mouse in vivo, as shown in experimental setup 466. Images 468 are captured of the flow, with individual particles used to track the flow (e.g., tracer particles). The present techniques are able to produce both two-dimensional velocity fields 470 and pressure fields 472 and three-dimensional velocity fields 476 and pressure fields 478.

Regarding surface currents, FIG. 4H depicts experimental results using sea surface temperature to predict flow along a sea's surface (e.g., in the Gulf of Mexico). Based on satellite images reflecting sea surface temperature, the present techniques are able to track changes in temperature (e.g., instead of individual particles) to determine predicted flows 480, 482.

Regarding 3D wakes again, FIG. 4I depicts experimental results to predict three-dimensional flows around a vehicle (e.g., a race car), as shown in setup 484. Conventional techniques require lots of data in order to generate complete three-dimensional flows 486. By contrast, the present techniques are able to receive data 488 from a limited number of slices (e.g., 3 slices, 5 slices, 10 slices) and use that data to reconstruct the three-dimensional flow 490. This can dramatically reduce the cost and time required to experimentally determine the 3D wake for a vehicle, as the vehicle may only need to spend several hours (or less) in a wind tunnel, rather than days or weeks, to determine the data for the slices instead of trying to directly capture the entire 3D flow.

Regarding other types of fluid, FIG. 4J depicts experimental results of airflow over a cup of espresso (e.g., as the espresso is being made). In particular, as the setup 492 shows, multiple cameras (e.g., 6 cameras) were used to captures images 494 of the temperature of the air surrounding a recently-made cup of espresso (e.g., using a tomographic background-oriented Schlieren experiment). The present techniques then utilized the images to reconstruct velocity flows 496 and pressure flows 498 for the air surrounding the espresso cup over time.

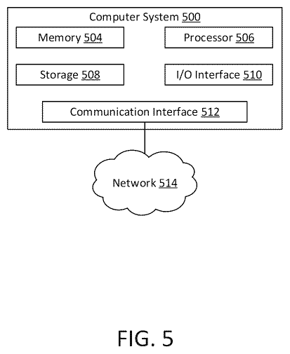

FIG. 5 illustrates an example computer system 500 that may be utilized to implement one or more of the devices and/or components discussed herein, such as the computing device 102 and/or the database 104. In particular embodiments, one or more computer systems 500 perform one or more steps of one or more methods described or illustrated herein. In particular embodiments, one or more computer systems 500 provide the functionalities described or illustrated herein. In particular embodiments, software running on one or more computer systems 500 performs one or more steps of one or more methods described or illustrated herein or provides the functionalities described or illustrated herein. Particular embodiments include one or more portions of one or more computer systems 500. Herein, a reference to a computer system may encompass a computing device, and vice versa, where appropriate. Moreover, a reference to a computer system may encompass one or more computer systems, where appropriate.

This disclosure contemplates any suitable number of computer systems 500. This disclosure contemplates the computer system 500 taking any suitable physical form. As example and not by way of limitation, the computer system 500 may be an embedded computer system, a system-on-chip (SOC), a single-board computer system (SBC) (such as, for example, a computer-on-module (COM) or system-on-module (SCAM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computer system 500 may include one or more computer systems 500; be unitary or distributed; span multiple locations; span multiple machines; span multiple data centers; or reside in a cloud, which may include one or more cloud components in one or more networks. Where appropriate, one or more computer systems 500 may perform without substantial spatial or temporal limitation one or more steps of one or more methods described or illustrated herein. As an example and not by way of limitation, one or more computer systems 500 may perform in real time or in batch mode one or more steps of one or more methods described or illustrated herein. One or more computer systems 500 may perform at different times or at different locations one or more steps of one or more methods described or illustrated herein, where appropriate.

In particular embodiments, computer system 500 includes a processor 506, memory 504, storage 508, an input/output (I/O) interface 510, and a communication interface 512. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 506 includes hardware for executing instructions, such as those making up a computer program. As an example and not by way of limitation, to execute instructions, the processor 506 may retrieve (or fetch) the instructions from an internal register, an internal cache, memory 504, or storage 508; decode and execute the instructions: and then write one or more results to an internal register, internal cache, memory 504, or storage 508. In particular embodiments, the processor 506 may include one or more internal caches for data, instructions, or addresses. This disclosure contemplates the processor 506 including any suitable number of any suitable internal caches, where appropriate. As an example and not by way of limitation, the processor 506 may include one or more instruction caches, one or more data caches, and one or more translation lookaside buffers (TLBs). Instructions in the instruction caches may be copies of instructions in memory 504 or storage 508, and the instruction caches may speed up retrieval of those instructions by the processor 506. Data in the data caches may be copies of data in memory 504 or storage 508 that are to be operated on by computer instructions; the results of previous instructions executed by the processor 506 that are accessible to subsequent instructions or for writing to memory 504 or storage 508; or any other suitable data. The data caches may speed up read or write operations by the processor 506. The TLBs may speed up virtual-address translation for the processor 506. In particular embodiments, processor 506 may include one or more internal registers for data, instructions, or addresses. This disclosure contemplates the processor 506 including any suitable number of any suitable internal registers, where appropriate. Where appropriate, the processor 506 may include one or more arithmetic logic units (ALUs), be a multi-core processor, car include one or ore processors 506. Although this disclosure describes and illustrates a particular processor, this disclosure contemplates any suitable processor.

In particular embodiments, the memory 504 includes main memory for storing instructions for the processor 506 to execute or data for processor 506 to operate on. As an example, and not by way of limitation, computer system 500 may load instructions from storage 508 or another source (such as another computer system 500) to the memory 504. The processor 506 may then load the instructions from the memory 504 to an internal register or internal cache. To execute the instructions, the processor 506 may retrieve the instructions from the internal register or internal cache and decode them. During or after execution of the instructions, the processor 506 may write one or more results (which may be intermediate or final results) to the internal register or internal cache. The processor 506 may then write one or more of those results to the memory 504. In particular embodiments, the processor 506 executes only instructions in one or more internal registers or internal caches or in memory 504 (as opposed to storage 508 or elsewhere) and operates only on data in one or more internal registers or internal caches or in memory 504 (as opposed to storage 508 or elsewhere). One or more memory buses (which may each include an address bus and a data bus) may couple the processor 506 to the memory 504. The bus may include one or more memory buses, as described in further detail below. In particular embodiments, one or more memory management units (MMUs) reside between the processor 506 and memory 504 and facilitate accesses to the memory 504 requested by the processor 506. In particular embodiments, the memory 504 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 504 may include one or more memories 504, where appropriate. Although this disclosure describes and illustrates particular memory implementations, this disclosure contemplates any suitable memory implementation.

In particular embodiments, the storage 508 includes mass storage for data or instructions. As an example and not by way of limitation, the storage 508 may include a hard disk drive (HDD), a floppy disk drive, flash memory, an optical disc, a magneto-optical disc, magnetic tape, or a Universal Serial Bus (USB) drive or a combination of two or more of these. The storage 508 may include removable or non-removable (or fixed) media, where appropriate. The storage 508 may be internal or external to computer system 500, where appropriate. In particular embodiments, the storage 508 is non-volatile, solid-state memory. In particular embodiments, the storage 508 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or combination of two of these. This disclosure contemplates mass storage 508 taking any suitable physical form. The storage 508 may include one or more storage control units facilitating communication between processor 506 and storage 508, where appropriate. Where appropriate, the storage 508 may include one or more storages 508. Although this disclosure describes and illustrates particular storage, this disclosure contemplates any suitable storage.

In particular embodiments, the I/O Interface 510 includes hardware, software, or both, providing one or more interfaces for communication between computer system 500 and one or more I/O devices. The computer system 500 may include one or more of these I/O devices, where appropriate. One or more of these I/O devices may enable communication between a person (i.e., a user) and computer system 500. As an example and not by way of limitation, an I/O device may include a keyboard, keypad, microphone, monitor, screen, display panel, mouse, printer, scanner, speaker, still camera, stylus, tablet, touch screen, trackball, video camera, another suitable I/O device or a combination of two or more of these. An I/O device may include one or more sensors. Where appropriate, the I/O Interface 510 may include one or more device or software drivers enabling processor 506 to drive one or more of these I/O devices. The I/O interface 510 may include one or more I/O interfaces 510, where appropriate. Although this disclosure describes and illustrates a particular I/O interface, this disclosure contemplates any suitable I/O interface or combination of I/O interfaces.

In particular embodiments, communication interface 512 includes hardware, software, or both providing one or more interfaces for communication (such as, for example, packet-based communication) between computer system 500 and one or more other computer systems 500 or one or more networks 514. As an example and not by way of limitation, communication interface 512 may include a network interface controller (NIC) or network adapter for communicating with an Ethernet or any other wire-based network or a wireless NIC (WNIC) or wireless adapter for communicating with a wireless network, such as a Wi-Fi network. This disclosure contemplates any suitable network 514 and any suitable communication interface 512 for the network 514. As an example and not by way of limitation, the network 514 may include one or more of an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 500 may communicate with a wireless PAN (WPAN) (such as, for example, a Bluetooth® WPAN), a WI-FI network, a WI-MAX network, a cellular telephone network (such as, for example, a Global System for Mobile Communications (GSM) network), or any other suitable wireless network or a combination of two or more of these. Computer system 500 may include any suitable communication interface 512 for any of these networks, where appropriate. Communication interface 512 may include one or more communication interfaces 512, where appropriate. Although this disclosure describes and illustrates a particular communication interface implementations, this disclosure contemplates any suitable communication interface implementation.

The computer system 502 may also include a bus. The bus may include hardware, software, or both and may communicatively couple the components of the computer system 500 to each other. As an example and not by way of limitation, the bus may include an Accelerated Graphics Port (AGP) or any other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-PIN-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local bus (VLB), or another suitable bus or a combination of two or more of these buses. The bus may include one or more buses, where appropriate. Although this disclosure describes and illustrates a particular bus, this disclosure contemplates any suitable bus or interconnect.

Herein, a computer-readable non-transitory storage medium or media may include one or more semiconductor-based or other types of integrated circuits (ICs) (e.g., field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, features, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

1. A method comprising:
receiving a plurality of microfluidic images of fluid flow within a fluid channel at a plurality of times;
analyzing, with a machine learning model, the plurality of microfluidic images to predict at least two fields for predicted fluid flow within the fluid channel, the at least two fields selected from the group consisting of a velocity field, a pressure field, and/or a stress field;
calculating a loss measure for the at least two fields based on at least two of physical fluid flow constraints, boundary condition constraints for fluid flow within the fluid channel, and data mismatch constraints between the predicted fluid flow and the plurality of microfluidic images; and
updating the machine learning model based on the loss value.

2. The method of claim 1, wherein the at least two fields are two-dimensional fields for the predicted fluid flow.

3. The method of claim 1, wherein the at least two fields are three-dimensional fields for the predicted fluid flow.

4. The method of claim 1, wherein the boundary condition constraints include a boundary condition measure computed based on compliance of the predicted fluid flow with a predetermined boundary condition.

5. The method of claim 4, wherein the predetermined boundary condition is selected from the group consisting of a slip boundary condition and a non-slip boundary condition.

6. The method of claim 1, wherein the physical fluid flow constraints include a physical conservation measure computed to measure compliance of the predicted fluid flow with fluid dynamic flow constraints.

7. The method of claim 6, wherein the fluid dynamic flow constraints include an optical flow constraint.

8. The method of claim 6, wherein the physical conservation measure is computed at a predetermined set of coordinates within the predicted fluid flow.

9. The method of claim 1, wherein the machine learning model is a fully-connected neural network.

10. The method of claim 1, wherein the microfluidic images are two-dimensional images of the fluid channel.

11. The method of claim 1, wherein the microfluidic images are three-dimensional images of the fluid channel.

12. The method of claim 1, wherein the microfluidic images are successive images captured by a video camera.

13. The method of claim 1, wherein the fluid is blood and the microfluidic images depict at least one of individual blood vessels and/or individual platelets.

14. A system comprising:
a processor; and
a memory storing instructions which, when executed by processor, cause the processor to:
receive a plurality of microfluidic images of fluid flow within a fluid channel at a plurality of times;
analyze, with a machine learning model, the plurality of microfluidic images to predict at least two fields for predicted fluid flow within the fluid channel, the at least two fields selected from the group consisting of a velocity field, a pressure field, and/or a stress field;
calculate a loss measure for the at least two fields based on at least two of physical fluid flow constraints, boundary condition constraints for fluid flow within the fluid channel, and data mismatch constraints between the predicted fluid flow and the plurality of microfluidic images; and
update the machine learning model based on the loss value.

15. The system of claim 14, wherein the at least two fields are two-dimensional fields for the predicted fluid flow.

16. The system of claim 14, wherein the at least two fields are three-dimensional fields for the predicted fluid flow.

17. The system of claim 14, wherein the boundary condition constraints include a boundary condition measure computed based on compliance of the predicted fluid flow with a predetermined boundary condition.

18. The system of claim 17, wherein the predetermined boundary condition is selected from the group consisting of a slip boundary condition and a non-slip boundary condition.

19. The system of claim 14, wherein the physical fluid flow constraints include a physical conservation measure computed based on compliance of the predicted fluid flow with fluid dynamic flow constraints.

20. The system of claim 19, wherein the fluid dynamic flow constraints include an optical flow constraint.
